(19) United States
(12) Patent Application Publication
(10) Pub. No.: US 2008/0082144 A1
(43) Pub. Date: Apr. 3, 2008

(54) UNIVERSAL USB-BASED TELEMETRY RF HEAD

(76) Inventors: James Marcotte, Arden Hills, MN (US); Christopher M. Petersen, Ham Lake, MN (US)

Correspondence Address: MEDTRONIC, INC., 710 MEDTRONIC PARKWAY NE, MINNEAPOLIS, MN, 55432-9924, US

(21) Appl. No.: 11/537,014

(22) Filed: Sep. 29, 2006

(51) Int. Cl. A61N 1/00

(52) U.S. Cl. 607/60; 607/31; 607/32

(57) ABSTRACT

A telemetry module connects to a computing device in order to perform functions related to programming and interaction with an implantable medical device (IMD). The telemetry module manages communication between the computing device and the IMD, and ensures that instructions provided by the computing device for programming the IMD are valid and safe before those instructions are executed.

### BACKGROUND OF THE INVENTION

The present invention relates to systems and devices for interrogating and programming implantable medical devices (IMDs).

IMDs for producing a therapeutic result in a patient are well known, and include implantable cardiac pacemakers, cardioverters, defibrillators, drug infusion pumps, neurostimulators, and other devices. Many of these devices provide an electrical output or otherwise contain electrical circuitry to perform their intended function.

An external device, commonly known as a programmer, is typically used to interface with an IMD using telemetry. Such an external device can be used for a number of tasks associated with an IMD. Examples of tasks include obtaining information about the condition, state or status of the IMD, obtaining information about the patient such as information related to the therapy intended to be provided by the IMD, transmitting information to the IMD specifying the therapy parameters to be provided by the IMD, and transmitting new or updated maintenance information concerning the operation of the IMD. In short, an external programmer is intended to perform all necessary or desired communication functions with an IMD.

External programmers typically consist of several different components that perform several different functions, such as a telemetry module for conducting communications with the IMD according to an appropriate protocol, a user interface for receiving input from a user and displaying information, and others. In many cases, external programmers are relatively complex because of the need to provide specific and critical functionality for interaction with the IMD and with a user. This has resulted in specific, complex, and relatively expensive external programmers typically being developed individually for different IMDs, which consumes time and resources and adds to the overall cost of medical treatment.

General purpose computers, while containing the processing capability to implement many of the functions of an external programmer, present problems in medical device applications because of the lack of control that the medical device manufacturer has over the computer. The specific character, format, and program environment of the general purpose computer is not known to the medical device manufacturer, which is often not acceptable in a medical device programming application because of the critical nature of interactions with the device.

An IMD programming solution that provides a versatile, relatively inexpensive device that maintains security and control over programming functions would be a useful improvement in the art.

### BRIEF SUMMARY OF THE INVENTION

The present invention is a telemetry module for connection to a computing device in order to perform functions related to programming and interaction with an implantable medical device (IMD). The telemetry module manages communication between the computing device and the IMD, and ensures that instructions provided by the computing device for programming the IMD are valid and safe before those instructions are executed. Generally, telemetry connection is a wireless data application system. In the context of the invention, wireless refers to the use of signal processing algorithms and coding techniques to create a data communication channel using RF or equivalent without a wireline.

### BRIEF DESCRIPTION OF THE DRAWINGS

FIG. 1 is an illustration of an implantable medical device system.

FIG. 2 is a diagram of a telemetry module according to an embodiment of the present invention.

FIG. 3A is a diagram illustrating a first example of a system configuration employing a telemetry module.

FIG. 3B is a diagram illustrating a second example of a system configuration employing a telemetry module.

FIG. 3C is a diagram illustrating a third example of a system configuration employing a telemetry module.

FIG. 3D is a diagram illustrating a fourth example of a system configuration employing a telemetry module.

FIG. 4 is a diagram illustrating the hardware associated with a telemetry module according to an embodiment of the present invention.

FIG. 5 is a block diagram illustrating the software framework utilized in a telemetry module according to an embodiment of the present invention.

FIG. 6A is a block diagram illustrating the software framework utilized in a telemetry module in a first configuration of an IMD system.

FIG. 6B is a block diagram illustrating the software framework utilized in a telemetry module in a second configuration of an IMD system.

FIG. 6C is a block diagram illustrating the software framework utilized in a telemetry module in a third configuration of an IMD system.

FIG. 6D is a block diagram illustrating the software framework utilized in a telemetry module in a fourth configuration of an IMD system.

FIG. 7A is a flow diagram illustrating an example of a process by which a computing device wishing to transmit programming instructions makes a secure, certified connection with a telemetry module.

FIG. 7B is a flow diagram illustrating the process by which a computing device executes transmissions to a telemetry module after the telemetry module has certified the connection with the computing device.

### DETAILED DESCRIPTION

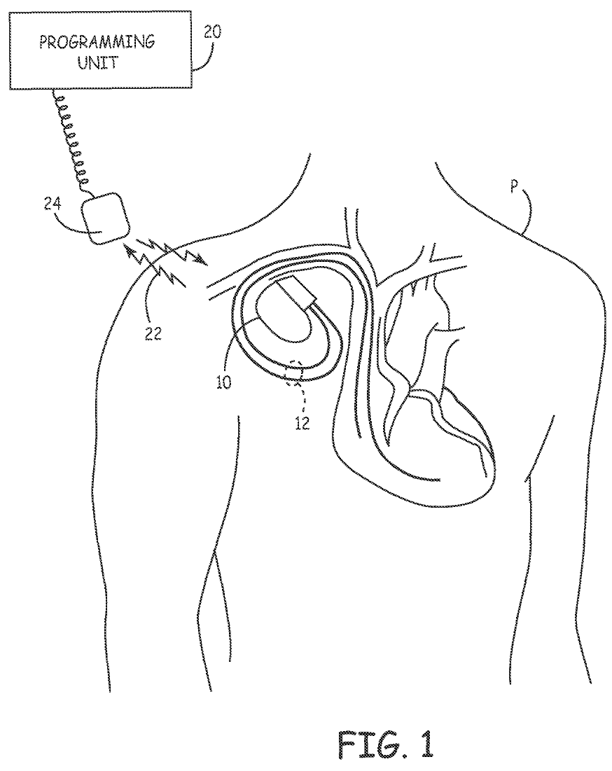

FIG. 1 is an illustration of an implantable medical device (IMD) system.

The IMD system includes IMD 10 (shown as a pacemaker in FIG. 1) which has been implanted in patient P. One or more leads, collectively identified by reference numeral 12, are electrically coupled to IMD 10 in a conventional manner. In the example of FIG. 1, in which IMD 10 is a pacemaker, leads 12 extend into the patient's heart via a vein. Other arrangements and configurations of leads 12 are known in the art for other types of IMDs.

Also depicted in FIG. 1 is external programming unit 20 for non-invasive communication with IMD 10 via telemetry channels 22. Telemetry head 24 is associated with programming unit 20 to perform two-way communication between IMD 10 and programming unit 20. Telemetry head 24 is positionable on the patient's body over the implant site of IMD 10, or may communicate with IMD 10 via distance telemetry, so that one or more antennae within the head to send RF signals to, and receive RF signals from, an antenna disposed in or on IMD 10, as is known in the art.

In existing IMD systems, programming unit 20 includes a number of functional components for storing and executing instructions related to communication with IMD 10, programming of IMD 10, and processing of data received from IMD 10. In most systems, these components are designed specifically for IMD 10 with which programming unit 20 is designed to operate. However, according to the principles of the present invention, the IMD system shown in FIG. 1 can utilize a modified telemetry head 24 that includes functionality that allows programming unit 20 to either be simplified or eliminated, so that a more generic computing device can be used for some of the functions that were previously provided by programming unit 20. Telemetry head 24 is configured to perform certification functions to ensure that interactions with the generic computing device are as secure and reliable as were interactions with a specifically designed programming unit.

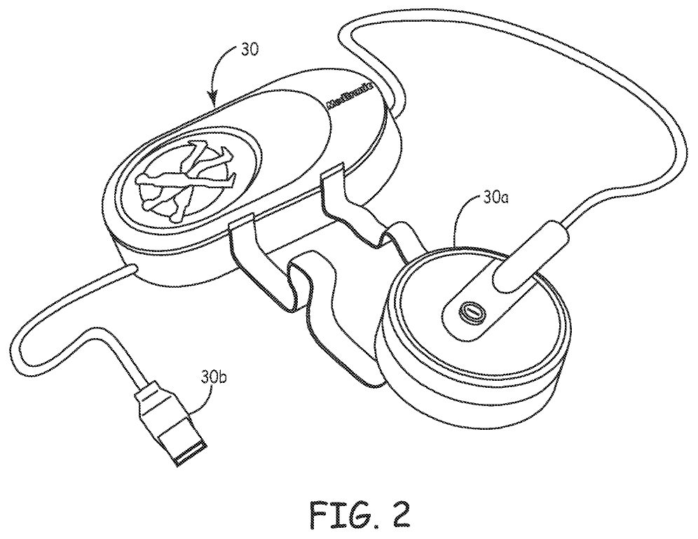

FIG. 2 is a diagram of telemetry module 30 according to an embodiment of the present invention. Telemetry module 30 includes telemetry head 30a and interface 30b, which may be a universal serial bus (USB) interface in one embodiment (which is shown in FIG. 2).

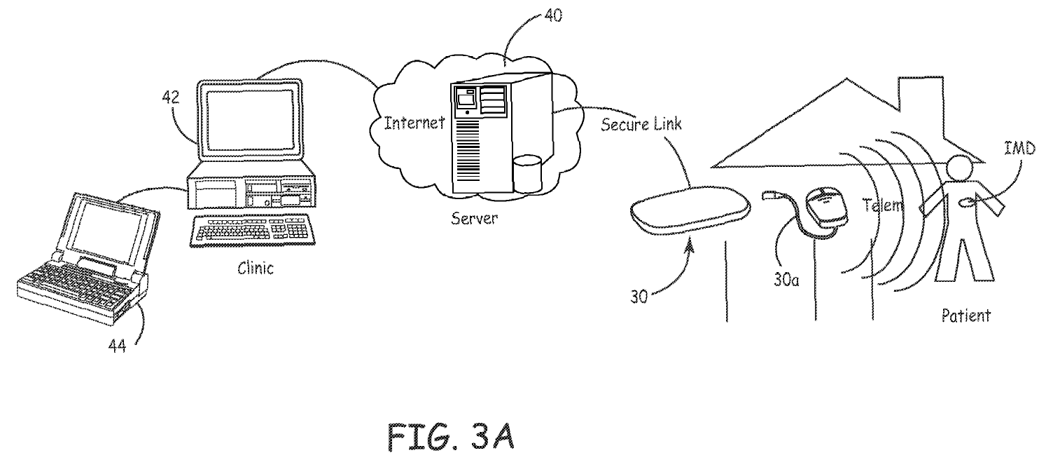

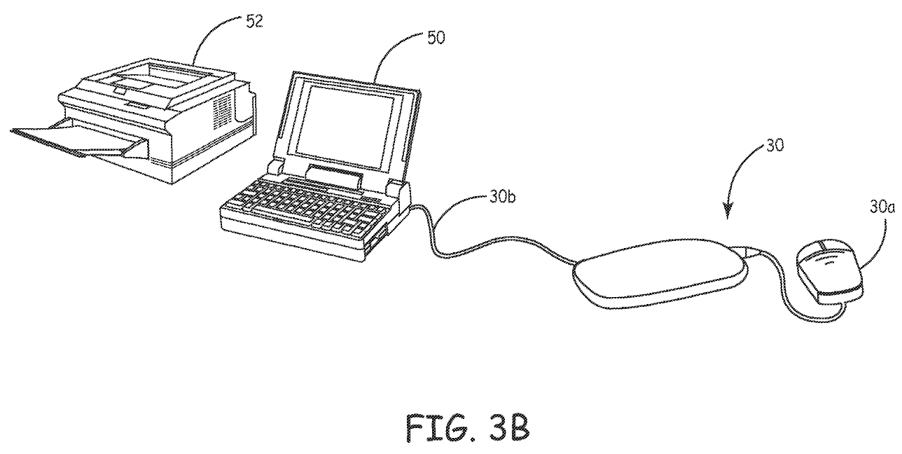

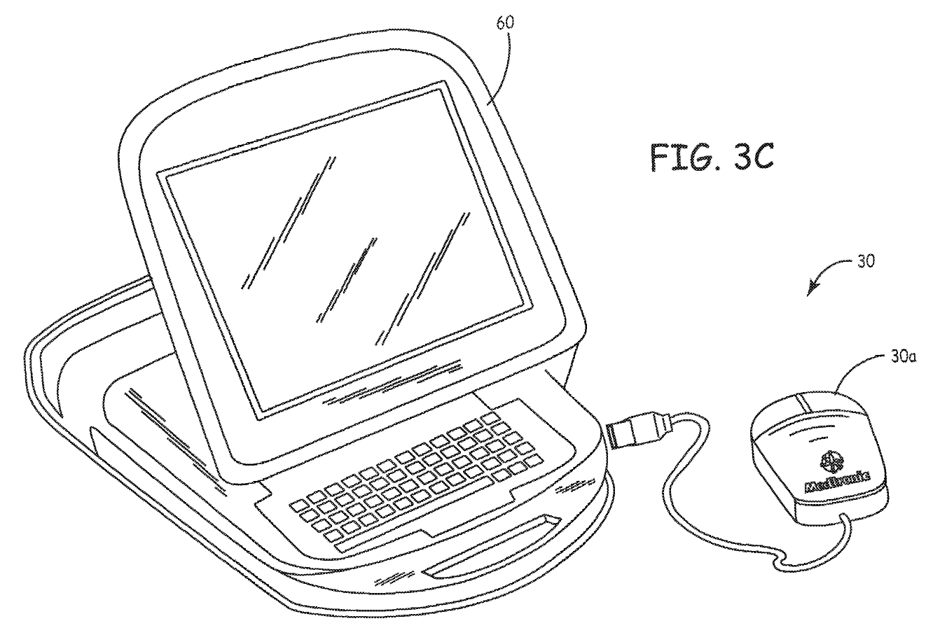

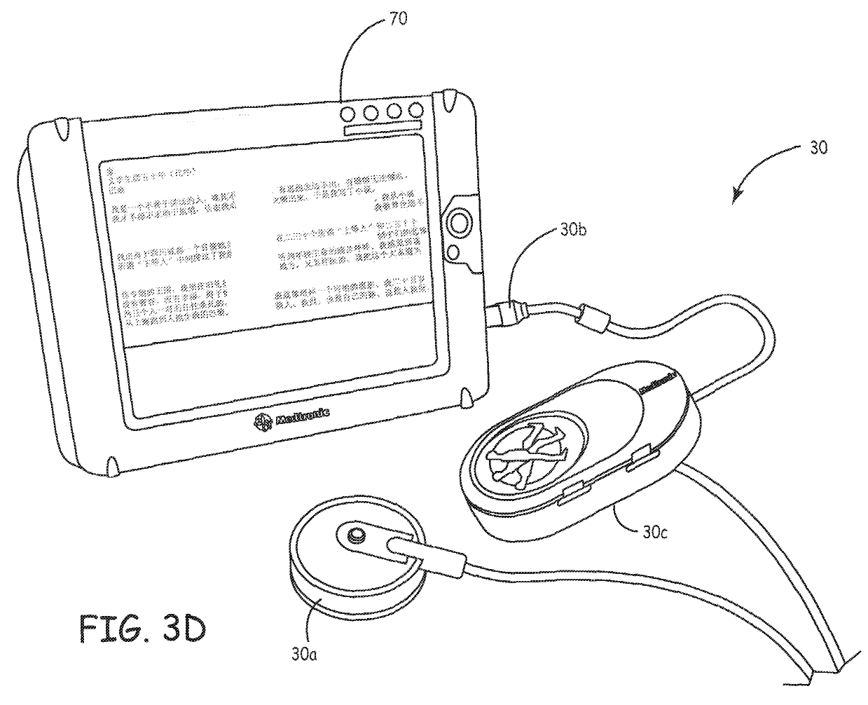

FIG. 3A is a diagram illustrating a first example of a system configuration employing telemetry module 30. In this system, telemetry module 30 is employed in the home of a patient having an IMD. Telemetry head 30a communicates with the IMD to interrogate and/or program the device. Telemetry module 30 communicates over a secure link with a clinic via controlled server 40, which may be a CareLink® network server manufactured by Medtronic, Inc., for example. Server 40 is coupled to computer 42 at the clinic via an internet connection or a similar type of connection. Computer 42 may be connected directly or via a network to various peripheral equipment, such as printer 44.

In this configuration, remote programming of a patient's IMD may be performed, such as by the Medtronic CareLink® network. Telemetry module 30 serves as an in-home monitor, or may be connected to additional hardware to provide the in-home monitor, which is designed to be used by an unskilled person (e.g., the patient). Direct interaction with the patient is therefore limited appropriately. The monitor may function to monitor the IMD and to communicate data collected from the IMD to server 40 at a remote location, such as via a telephone line or by other connections or links. Based on the data collected and communicated, appropriate instructions for programming the IMD may be selected at a clinic via computer 42 that is communicatively coupled to server 40, such as by medical personnel operating appropriate software on computer 42, or by automatic selection of the software itself). The selected instructions are then transmitted back to telemetry module 30 via server 40, so that programming of the IMD can be performed.

FIG. 3B is a diagram illustrating a second example of a system configuration employing telemetry module 30. In this configuration, telemetry module 30 is coupled to computer 50 via interface 30b (which may be a USB interface, for example). Telemetry head 30a is operable to communicate wirelessly with an IMD, interrogating the IMD and passing the retrieved data to computer 50, transmitting signals for programming the IMD based on instructions received from computer 50, or both. Computer 50 may be a portable personal computer of some sort, a personal digital assistant (PDA), or any other type of computing device. Computer 50 may also be connected directly or via a network to various peripheral equipment, such as printer 52.

FIG. 3C is a diagram illustrating a third example of a system configuration employing telemetry module 30. In this configuration, telemetry head 30a is coupled to programming device 60, which integrally includes the functionality of the remaining portion of telemetry module 30. Programming device 60 is similar to existing programmers in this embodiment, executing software for communicating with an IMD through telemetry head 30a to interrogate the IMD and transmit programming signals to the IMD.

FIG. 3D is a diagram illustrating a fourth example of a system configuration employing telemetry module 30. In this configuration, telemetry module 30 is coupled to tablet computer 70 (or a similar computing device) via USB interface 30b. Telemetry head 30a is operable to communicate wirelessly with an IMD, interrogating the IMD and passing the retrieved data to computer 70, and transmitting signals for programming the IMD. Telemetry module 30 is divided into telemetry processing unit 30c and telemetry head 30a. USB interface 30b is about 0.5 meters long in one embodiment. Telemetry processing unit 30c contains the electronics and logic of telemetry module 30, and receives power from computer 70 via USB interface 30b at 0.5 Amperes and 5 Volts. Telemetry head 30a requires higher voltage to perform RF telemetry, and so telemetry processing unit 30c transforms the power to a higher voltage for use by telemetry head 30a. The interface between telemetry processing unit 30c and telemetry head 30a is about 2 meters long in one embodiment.

In one embodiment, the system includes one or more of the following features:

The application software executed by computer 70 is stored in a compact flash card (or other media) that is physically blocked from being accessed without a tool. Further, power is provided to telemetry module 30 from computer 70 via USB interface 30b. Further, more data is handled via Unicode resource DLLs in order to support various languages, such as Chinese. Additionally, a “dual emergency key” feature is provided for pacing applications, which requires the simultaneous pressing of two buttons (either hardware-based buttons, software-based buttons, or a combination of the two, for example) in order to activate emergency pacing (in order to avoid inadvertent activation of emergency pacing).

In some embodiments, telemetry module 30 may itself offer a simplified or basic application, in addition to supporting a computing device-based application. For example, a disconnected telemetry module could operate to verify basic health parameters and IMD functions, while more complex issues that are identified which require more information or programming capability could require connection to a computing device. In one embodiment, the basic status information could be simply red and green lights (indicating either an “OK” or “more information required” status), or a more detailed on-board display, depending on the desired environment for use.

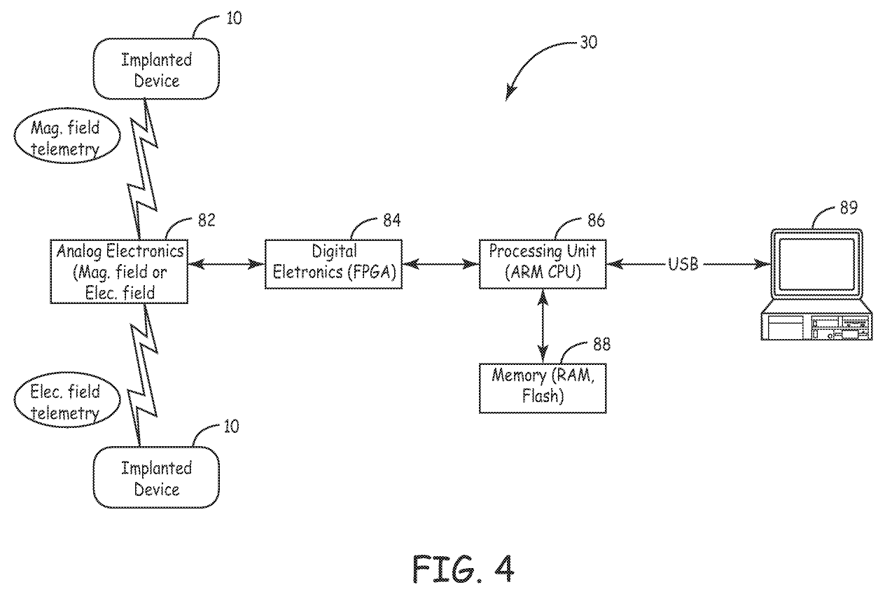

FIG. 4 is a diagram illustrating the hardware associated with telemetry module 30. Telemetry module 30 is communicatively coupled to IMD 10. This communicative connection may be made by a variety of telemetry schemes, such as electric field telemetry, magnetic field telemetry, or others (both alternatives are illustrated in FIG. 4, by showing two IMDs 10, one communicating with electric field telemetry and the other communicating with magnetic field telemetry). Analog electronics 82 provide the appropriate physical interface for the telemetry scheme that is used. Digital electronics 84 are connected to analog electronics 82, and are configured to support the telemetry scheme that is employed. In one embodiment, digital electronics 84 are implemented in a dynamically configurable field programmable gate array (FPGA). Digital electronics 84 are connected to processing unit 86, which is a scalable ARM® central processing unit (CPU) in one embodiment. Processing unit 86 is connected to memory 88, which may be a flash read only memory (ROM), a random access memory (RAM), or another type of memory known in the art. Processing unit 86 is coupled to computer 89 via an interface, which may be an industry standard interface such as a universal serial bus (USB) interface in one embodiment.

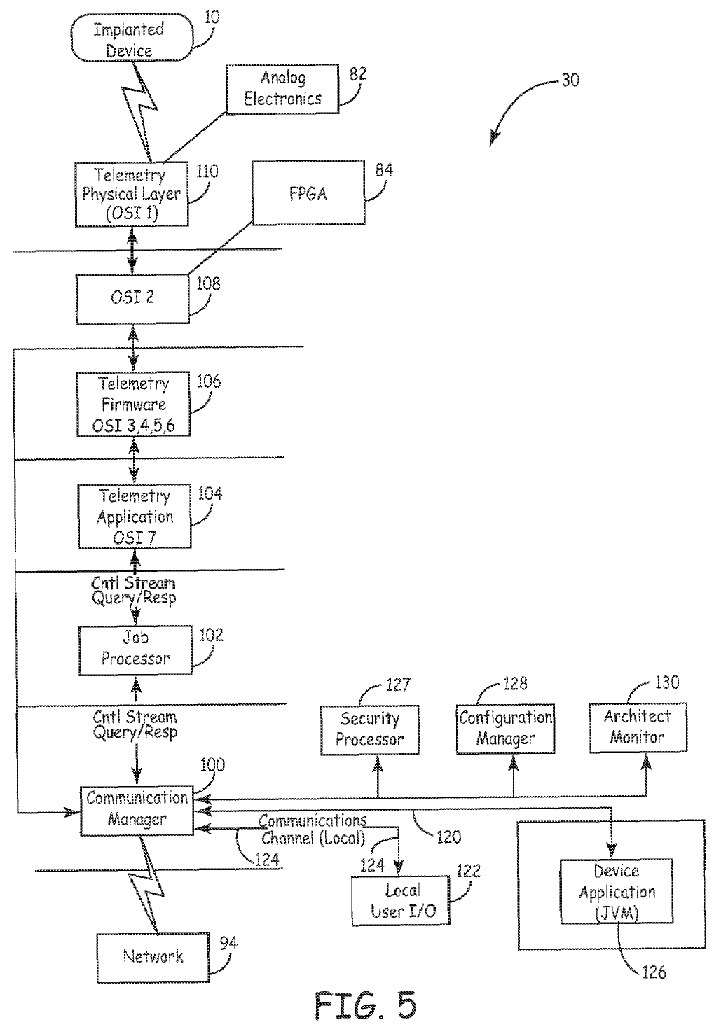

In operation, computer 89 executes an application that provides a user interface for interaction with IMD 10, similar to special purpose programming units that currently exist. Telemetry module 30 provides communication capability between computer 89 and IMD 10 by appropriate telemetry, which depends on the type of IMD 10 that is employed. Telemetry module 30 also is responsible for insuring that the interactions between computer 89 and IMD 10 are valid and safe. This is an important feature of telemetry module 30, due to the many possible forms, functions, capabilities and security associated with computer 89. For example, computer 89 may be a programming unit provided by the same manufacturer that manufactured IMD 10. Computer 89 may also be a programming unit provided by a different manufacturer. Computer 89 may alternatively be a general purpose computer executing an application that allows computer 89 to function as a programming unit, either locally or remotely (e.g., over the Internet/World Wide Web), or may be a personal digital assistant (PDA) or other portable device executing a similar type of application. This capability afforded by telemetry module 30 enables a variety of equipment configurations to perform the function of IMD programming, which can result in significant cost savings and/or service enhancements for patients and medical facilities. The details of the functions performed by telemetry module 30 are discussed below with respect to the software shown in FIG. 5.

FIG. 5 is a block diagram illustrating the software framework utilized in telemetry module 30 of the present invention, shown with reference to the Open Systems Interconnection (OSI) seven-layer model. The diagram shows telemetry module 30 providing communication between IMD 10 and device application 126 (which could reside on a programming unit, a general purpose computer, or other computing/communication components). Telemetry module 30 includes a number of functional components, including communication manager 100, job processor 102, telemetry application 104 (OSI layer 7), telemetry firmware 106 (OSI layers 3, 4, 5 and 6), telemetry data link layer 108 (OSI layer 2), and telemetry physical layer 110 (OSI layer 1) (although it should be understood throughout the discussion below that these components and layers may be combined or eliminated in some embodiments).

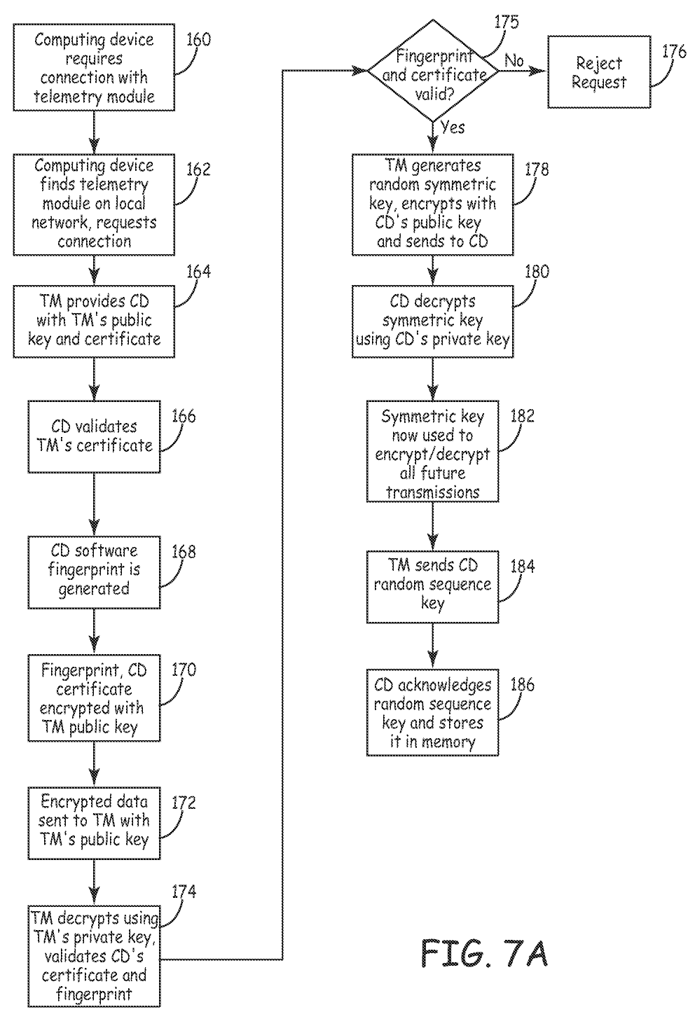

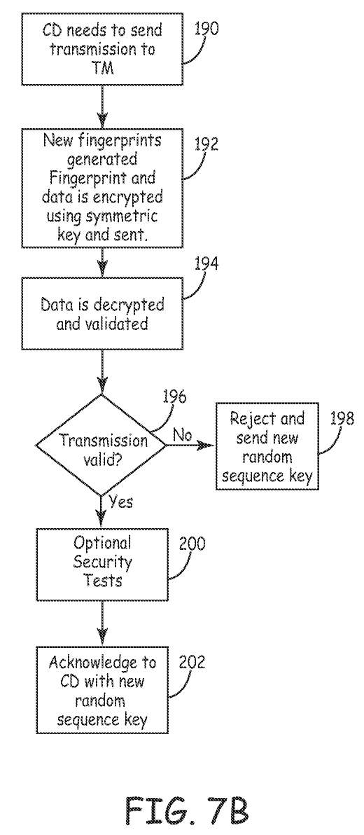

Communication manager 100 is responsible for managing communications between telemetry module 30 and device application 126, to acquire information from device application 126 that will be used to program IMD 10, and to provide information to device application 126 representative of the operation and/or status of IMD 10, the patient in whom IMD 10 is implanted, or both. Communication manager 100 communicates over communication channel 120 (which in one example is a USB interface), and also communicates information with local user input/output (I/O) interface 122 over local communication channel 124, as well as with network 94. Communication manager 100 also controls and/or monitors functions performed by job processor 102, telemetry application 104, telemetry firmware 106, and telemetry data link layer 108 based on data received over network communication channel 120 and/or local communication channel 124. Communication manager 100 also communicates with security processor 127, configuration manager 128 and architect monitor 130 (an optional component for capturing diagnostic information) to further control job processor 102, telemetry application 104, telemetry firmware 106, and telemetry data link layer 108. Security processor 127 provides cryptographic functions for communication manager 100, managing public/private key pairs, certificate chains of authority, and fingerprint generation and validation. The certification and security provided in the operation of telemetry module 30 is explained in detail below with respect to FIGS. 7A and 7B.

Job processor 102 is responsible for controlling the tasks performed by telemetry head 30. Telemetry head 30 may operate in a number of modes, such as a basic mode, an autonomous mode, a networked mode, a maintenance mode, or others. In one embodiment, these modes of operation are characterized as follows:

Basic Mode—In basic mode, low-level telemetry commands are exposed to device application 126, similar to the operation of existing programming units. Thus, basic mode is most appropriate when the connection between job processor 102 and device application 126 is a high speed, high reliability link, such as when telemetry module 30 is integrated in a programming unit or is locally connected to a programming unit.

Autonomous Mode—Autonomous mode includes the functions of Basic mode, and also allows device application 126 to assemble “jobs” and submit them to job processor 102 for execution. A job may include a series of commands, read requests, write requests, and real time data configuration commands, for example. Tasks that make up a job may be defined and executed conditionally, and macros may be employed so that tasks are executed based on the occurrence of certain events. Autonomous mode is suitable for use in scenarios where device application 126 is connected to telemetry module 30 via a network connection that may not be high speed or may have less reliability than a direct connection. This mode allows for local emergency activation of job processor 102 to automatically complete certain jobs (such as jobs that are deemed critical) if communication with device application 126 is interrupted.

Network Mode—Network mode includes the functions of Basic and Autonomous modes, and also allows communications between job processor 102 and other instruments. For example, job processor 102 may interact with a local automated external defibrillator (AED). This interaction could be used as an additional safety measure in appropriate situations, by requiring a patient to be connected to a defibrillator when IMD 10 is being reprogrammed.

Maintenance Mode—This mode provides development, test and debugging capability, when telemetry module 30 is not employed in an actual patient session.

In some embodiments, job processor 102 may be configured to accept remote programming via communications received from communication manager 100 over network communication channel 120, for example. Job processor 102 provides the capability to implement such programming in its control of tasks and its interaction with telemetry application 104, telemetry firmware 106, and telemetry data link layer 108. Job processor 102 may also interact with configuration manager 128 and communication manager 100 to implement an update of telemetry firmware 106 and telemetry data link layer 108 to support a new telemetry version.

Telemetry application 104 performs high level telemetry functions, such as automatic identification of device types (to identify IMD 10), real-time processing of data from IMD 10 (such as electrogram (EGM) signals and markers), and interrogation and programming of IMD 10. Telemetry application 104 may also perform a passthrough function, allowing OSI layer 7 services to be provided within device application 126 instead. These tasks are defined and executed in a manner that is specific to device application 126.

Telemetry firmware 106 includes the components of OSI layers 3, 4, 5 and 6. OSI layer 6 is the presentation layer. This layer provides independence from differences in data representation (e.g., encryption) by translating the data from application format to network format, and vice versa. Data is transformed into a form that telemetry application layer 104 can accept, and formats and encrypts data so that the data can be sent across a network, providing freedom from compatibility problems. This layer may also be called the syntax layer

OSI layer 5 is the session layer. This layer establishes, manages and terminates connections between applications. Specifically, the session layer sets up, coordinates, and terminates conversations, exchanges and dialogues between device IMD 10 and device application 126. The session layer coordinates the communication session and the connection between devices.

OSI layer 4 is the transport layer. This layer provides transparent transfer of data between IMD 10 and device application 126. The transport layer is responsible for end-to-end error recovery and flow control, to ensure complete data transfer. This layer breaks large messages from device application 126 down into a sequence of smaller data packets, and assembles packets received from IMD 10 into a message to be transmitted to device application 126.

OSI layer 3 is the network layer. This layer provides switching and routing capability, creating logical paths (also known as virtual circuits) for transmitting data from node to node. Routing and forwarding are functions of this layer, as well as addressing, internetworking, error handling, congestion control and packet sequencing.

Telemetry data link layer 108 forms OSI layer 2. In one embodiment, this layer is implemented in a FPGA, and is divided into a media access control (MAC) sublayer for controlling access to the transmission hardware, and a logical link control (LLC) for controlling frame synchronization and flow control and handling errors in the physical layer. Telemetry data link layer 108 encodes data packets and decodes data packets into bits, and also manages the transmission protocol.

Telemetry physical layer 100 forms OSI layer 1. This layer conveys the bit stream (electrical impulses, light signals or radio signals, for example) through the network at the electrical and mechanical level. This layer is implemented by analog electronics 82 and digital electronics 84 (including the FPGA) shown in FIG. 4, and provides the hardware for sending and receiving data on a carrier.

In operation, telemetry module 30 represents a trusted party in the IMD system, since it is under the control of the medical device manufacturer. In order to preserve its trusted status, any changes to its software may only be accomplished if they are sent from a trusted source. Telemetry module 30 will validate any request to supply a software update using cryptographic and authentication technology.

Similarly, telemetry module 30 communicates with a computing device that provides programming instructions using public/private and symmetric keys. The application software running on the computing device is isolated from other software on the computing device using a memory management unit (MMU), a virtual environment, or similar known technology. As part of its operation, the application software performs an analysis of its data and code space to create a fingerprint using cryptographic techniques. This fingerprint is encrypted and sent to telemetry module 30 along with possible commands or programming instructions. Telemetry module 30 decrypts the fingerprint and compares the fingerprint against a list of valid fingerprints for each possible device application. If the fingerprint is invalid, the commands or instructions are not executed. The fingerprint is periodically recalculated to verify the continued integrity of the application software.

The application software periodically executes performance tests to determine if the hardware has sufficient resources to properly maintain the device programming session. Should insufficient resources be available due to activity of other programs, the presence of a virus, or other factors, the software application will discontinue the device programming session and notify the user.

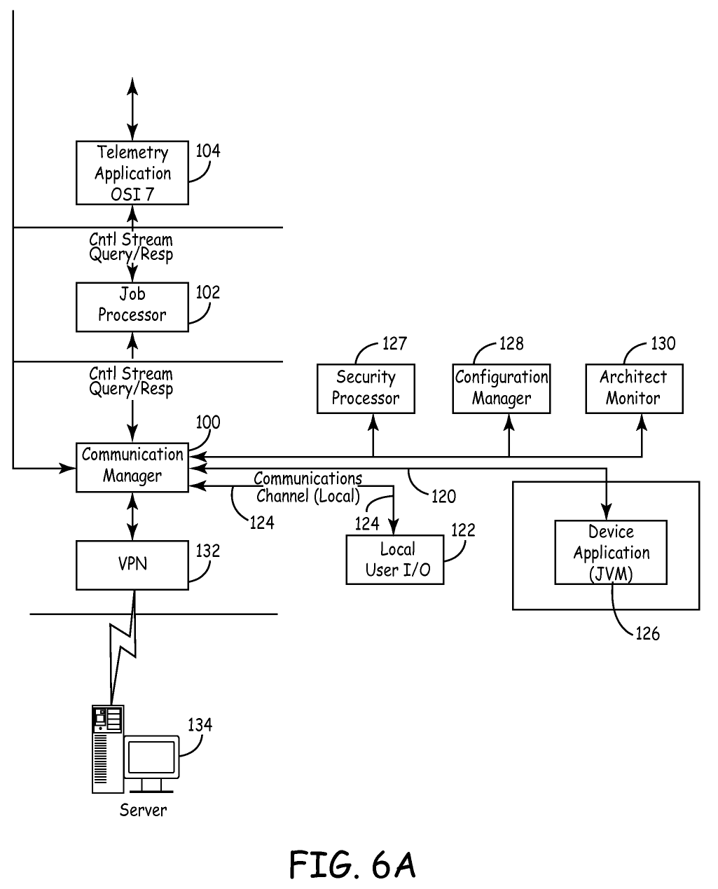

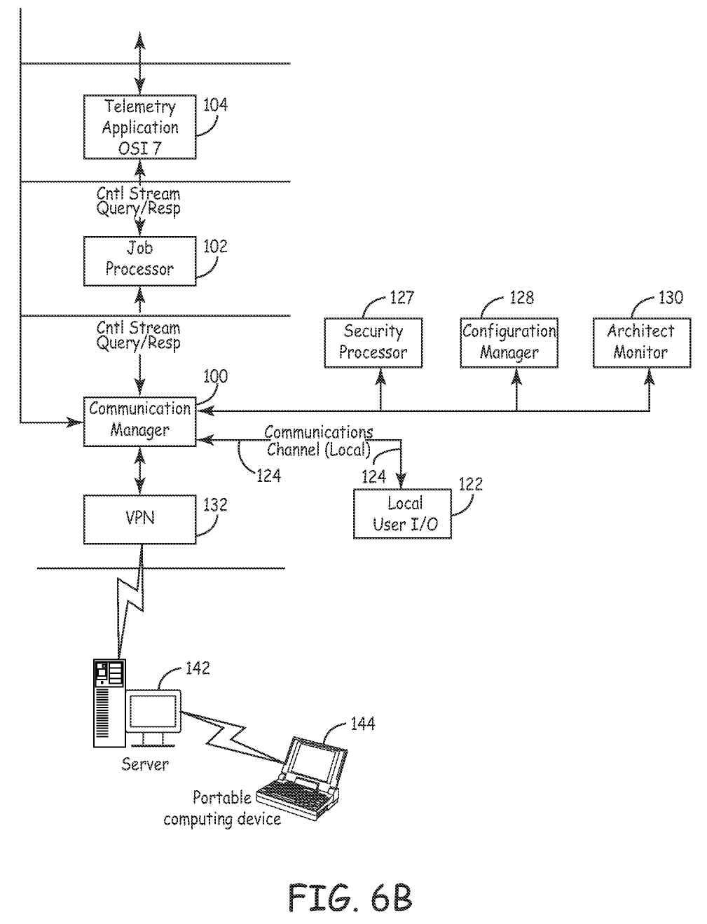

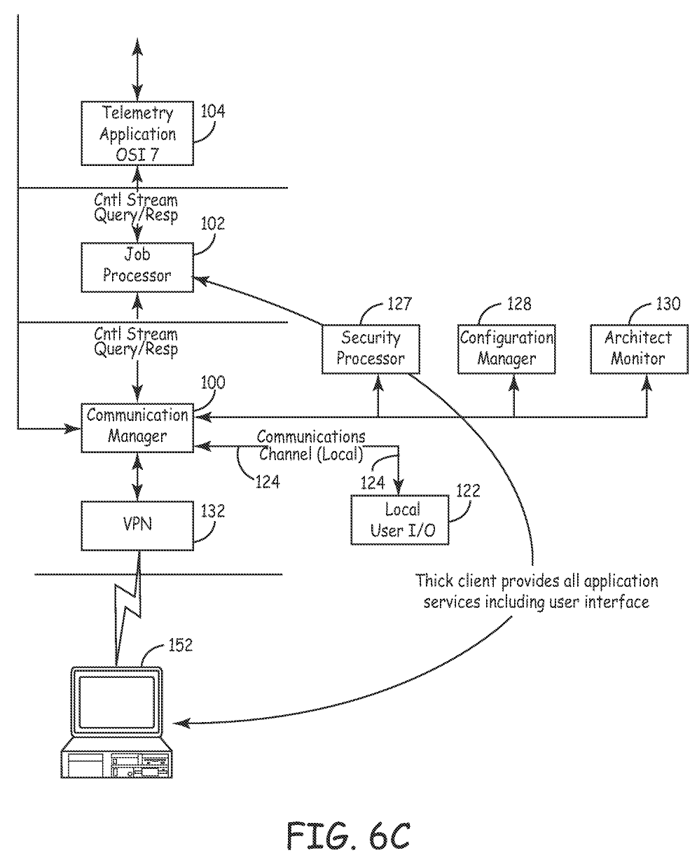

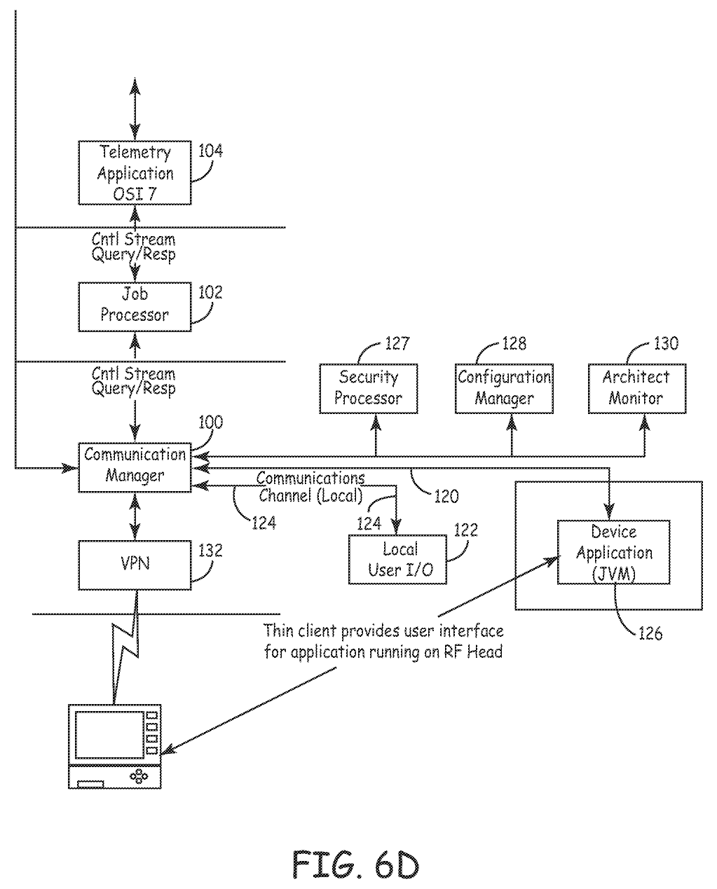

The functional components shown in FIG. 5 have been described above in a somewhat generic manner that is applicable to a number of different configurations of system components. The following discussion of FIGS. 6A, 6B, 6C and 6D will focus on specific examples of system configurations that may be employed according to various embodiments of the invention. These drawings only show components at the telemetry application layer 104 (OSI layer 7) and at higher levels, since the lower layers/levels in all of the configurations are the same.

FIG. 6A is a block diagram illustrating the software framework utilized in telemetry module 30 in a first configuration of an IMD system. The configuration shown in FIG. 6A is similar to the configuration shown in FIG. 5, except that network 94 shown in FIG. 5 is specifically identified as virtual private network (VPN) 132, which may be any network configuration having some level of administrative control over transmissions. In the example shown in FIG. 6A, VPN 132 is connected to server 134, which may be a CareLink® network server manufactured by Medtronic, Inc., for example. In this embodiment, remote programming of implanted device 10 may be provided via the CareLink® network. For example, a patient with IMD 10 may be at home with telemetry module 30 connected to (or formed as an integral part of) an in-home monitor (a constituent of VPN 132). In this embodiment, the in-home monitor may be designed to be used by an unskilled person (e.g., the patient), and therefore interaction with the patient is limited appropriately. The monitor may function to monitor IMD 10 and to communicate data collected from IMD 10 to CareLink® server 134 at a remote location, such as via a telephone line, an internet connection, or by other connections or links. Based on the data collected and communicated, appropriate instructions for programming implanted device 10 may be selected at the remote location (such as by medical personnel operating the device application software, or by automatic selection of the device application software) and transmitted back to the monitor and to communication manager 100 of telemetry module 30. Upon validation of the programming instructions, IMD 10 can then be programmed accordingly.

FIG. 6B is a block diagram illustrating the software framework utilized in telemetry module 30 in a second configuration of an IMD system. The configuration shown in FIG. 6B is similar to the configuration shown in FIG. 5, except that network 94 shown in FIG. 5 is specifically identified as virtual private network (VPN) 132 having server 142 in communication, and having portable computing device 144 communicatively coupled to server 142 to receive device data and provide programming instructions. In this embodiment, portable computing device 144 interacts with server 142 via a website (or other internet-type protocol). At least part of the device application (shown as 126 in FIG. 5) is stored and executed on either server 142 or portable computing device 144. For example, medical personnel located either in the vicinity of a telemetry head (e.g., within a room) or remotely from a telemetry head may operate a general purpose laptop computer or a personal digital assistant (PDA) executing a web browser-like application to provide programming instructions for an IMD, and to receive patient and device information from the IMD, via server 142 connected to VPN 132. The programming instructions provided from portable computing device 144 via server 142 are certified by telemetry module 30 before they are executed, to ensure that only valid instructions are performed to program IMD 10.

FIG. 6C is a block diagram illustrating the software framework utilized in telemetry module 30 in a third configuration of an IMD system. The configuration shown in FIG. 6C is similar to the configuration shown in FIG. 5, except that network 94 shown in FIG. 5 is specifically identified as virtual private network (VPN) 132 having application computer 152 as a constituent (or alternatively, application computer 152 may be the only constituent, in which case the designation of VPN 132 is unnecessary). In this embodiment, application computer 152 may be a programming device similar to programming devices that are currently in use, including the application software, user interface, and other features associated with such devices. Alternatively, application computer 152 may be a personal computer (PC) device programmed to execute software that is similar to the software executed by programming devices that are currently in use, or may be another type of device. Telemetry module 30 may be realized as a peripheral device connected to application computer 152 by an interface such as a USB interface, or may be integrated in some manner with application computer 152. Application computer 152 is a “thick” client with the capability to transmit complete sequences of programming instructions. Because application computer 152 is a trusted source of programming instructions in this embodiment, some of the security and certification features are performed internally in application computer 152, rather than by a component of telemetry module 30.

FIG. 6D is a block diagram illustrating the software framework utilized in telemetry module 30 in a fourth configuration of an IMD system. The configuration shown in FIG. 6D is similar to the configuration shown in FIG. 5, except that network 94 shown in FIG. 4 is specifically identified as virtual private network (VPN) 132 in communication with thin client component 156 (or alternatively, thin client component 156 may be the only constituent, in which case the designation of VPN 132 is unnecessary). In this embodiment, thin client 156 provides the user interface for operating device application 126 that runs on telemetry module. This configuration allows the device manufacturer to maintain control over the application program by storing it in telemetry module 30, while also allowing new and more modern hardware to be employed as the user interface for the application program via thin client 106.

Other configurations of components are of course possible as well, and the above-described examples are intended only to illustrate some of the configurations that can be achieved. These and other configurations are able to be used at least in part because of the safety and certification capability that is provided by telemetry module 30, which is described in detail below with respect to FIGS. 7A and 7B.

FIG. 7A is a flow diagram illustrating an example of a process by which a computing device wishing to transmit programming instructions makes a secure, certified connection with a telemetry module. Initially, as shown at step 160, the computing device recognizes that a connection with the telemetry module is required. The computing device then determines the relative location of the telemetry device on the communication network, and requests connection, as shown at step 162. The telemetry module (TM) then provides the computing device (CD) with the TM's public key and certificate, as shown at step 164. In one embodiment, the TM root certificate is installed when the TM is manufactured, along with a private/public key pair. The certificate and key pair are then stored in a secure processor (such as security processor 127 shown in FIG. 5), protected from reading or reverse engineering. In other embodiments, the private/public key pair is generated by the TM on an as-needed basis. The CD receives the TM's public key and certificate, and determines whether the public key and certificate are valid, at step 166. In some embodiment, the TM's public key and certificate may be presumed valid, such as when the TM is provided by a medical device manufacturer. After validation, the CD knows that the TM is valid, but the TM does not know whether the CD is certified to provide instructions.

The CD then generates a software fingerprint at step 168. The software fingerprint is based on the software structures, data structures, key values, etc. associated with the CD, and a change to any of these parameters would modify the software fingerprint. This allows detection of any alteration to the software due to malicious software (e.g., a virus, a worm, or others) running on the CD. The CD has no knowledge of what a valid fingerprint is, and so a valid software fingerprint cannot be reverse engineered. In one embodiment, the software fingerprint is generated using a one-way hash process, which prevents generation of a new fingerprint based only on a new random sequence key. The software fingerprint and a CD certificate are then encrypted with the TM's public key at step 170, and the encrypted data is transmitted to the TM with the TM's public key at step 172. Only the TM is able to decrypt this message, using the TM's private key, to validate the CD's certificate and fingerprint, as shown at step 174. In this step, the CD's certificate is used to validate its fingerprint. The CD's certificate is encrypted with the TM's public key, and thus, the TM knows whether the CD's certificate is valid (and also because the CD's certificate is digitally signed by a root authority that is under the control of the system manufacturer).

If the CD's fingerprint and certificate are determined (at decision step 175) by the TM to be invalid, the CD's request to connect with the TM is rejected, as shown at step 176. If the CD's fingerprint and certificate are determined by the TM to be valid, the TM generates a random symmetric key, encrypts that random symmetric key with the CD's public key, and sends it to the CD, as shown at step 178. The CD then decrypts the symmetric key using the CD's private key at step 180, so that the symmetric key is established as the key for encrypting and decrypting future transmissions, as shown at step 182.

During the communication session between the CD and the TM, the TM sends the CD a random sequence key, as shown at step 184. The CD acknowledges the random sequence key and stores it in its memory, as shown at step 186. The random sequence key allows a new and different fingerprint to be generated by the CD, so that copying and re-use of previously valid fingerprints is not possible.

FIG. 7B is a flow diagram illustrating the process by which the CD executes transmissions to the TM after the TM has validated the connection with the CD. Initially, the CD identifies a need to send a transmission to the TM at step 190. A new fingerprint is then generated by the CD using the symmetric key that had been previously sent to the CD by the TM, and the data for transmission to the TM is encrypted using the symmetric key as well, as shown at step 192. Changing the random sequence key prevents re-use of an old transmission, and prevents a software attack by re-using a previous fingerprint. The data transmitted by the CD is then decrypted and validated by the TM, as shown at step 194. The TM determines whether the transmission is valid at decision step 196, and if the transmission is invalid, the TM rejects the transmission and sends a new random sequence key to the CD, as shown at step 198. If the transmission is valid, the TM may perform additional optional security tests at step 200, to test for appropriate user credentials, vital signs within an appropriate range, the presence of certain auxiliary instrumentation, or other parameters. For example, certain programming instructions may only be allowed to be executed if defibrillator equipment is identified as being available, or certain other programming instructions may only be allowed to be executed if the patient's vital signs are in a specified range. The TM then sends an acknowledgement of the transmission to the CD, along with another new random sequence key for future transmissions, as shown at step 202.

The security associated with the software fingerprint allows the system to detect adulterated or otherwise improper software, such as software that has been altered due to a virus or worm, software that is out of date, software that is an incorrect version or is incompatible with certain aspects of the IMD or other components of the system, or others. In some embodiments, the software fingerprint is generated for only selected transmissions, such as transmissions that are critical to patient health, so that processing bandwidth is utilized efficiently. Similar procedures could be used to validate users of the IMD system, other equipment employed in the IMD system, new software acquired to update the system, or others.

The present invention, which can be implemented in a variety of embodiments and configurations, provides a telemetry module that is connectable to a computing device in order to perform the functions related to programming and interaction with an IMD that were typically performed by a specially designed programming unit in existing systems. These functions include receiving programming instructions from the computing device and certifying the safety of those instructions before performing the instructed programming (due to the potentially hazardous environment of general purpose computing devices), and converting data transmitted by an IMD into a format that is usable for a device application being executed on the computer device. This capability allows existing equipment that is already in use at a medical facility to be used, with appropriate software and firmware, as a programming unit, which could potentially allow IMDs to be utilized in environments and markets where they were not previously available.

Although the present invention has been described with reference to preferred embodiments, workers skilled in the art will recognize that changes may be made in form and detail without departing from the spirit and scope of the invention. For example, one skilled in the art will recognize that other types of medical devices, in addition to the examples described herein, can be employed in various embodiments while practicing the principles of the invention.

What is claimed is:

1. A telemetry module comprising:
telemetry electronics for communicating first signals with an implantable medical device (IMD);
an interface for communicating second signals with a computing device; and
a processing unit comprising software for controlling communications between the IMD and the computer device via the telemetry electronics and the interface, for certifying the validity of instructions for programming the IMD communicated by the computing device, and for enabling performance of the instructions for programming the IMD only after the instructions are certified as valid.

2. The telemetry module of claim 1, wherein the interface comprises a universal serial bus (USB) interface.

3. The telemetry module of claim 1, wherein the telemetry module further comprises an input/output (I/O) interface for displaying information related to the status of the IMD and/or inputting programming instructions.

4. The telemetry module of claim 1, wherein the telemetry module further comprises application software for executing the instructions for programming the IMD.

5. The telemetry module of claim 1, wherein the software for certifying the validity of the instructions for programming the IMD executes an encryption scheme to certify the validity of the instructions.

6. The telemetry module of claim 5, wherein the software for certifying the validity of the instructions for programming the IMD includes:
software for determining whether a software fingerprint of the computing device is valid.

7. The telemetry module of claim 6, wherein the software for determining whether a software fingerprint of the computing device is valid is operable to:
provide a public key associated with the telemetry module to the computing device;
receive the software fingerprint of the computing device encrypted with the public key;
decrypt the software fingerprint of the computing device with a private key associated with the telemetry module; and
determine whether the software fingerprint is valid.

8. The telemetry module of claim 6, wherein transmissions between the telemetry module and the computing device are encrypted and decrypted with a random symmetric key that is generated after determining that the software fingerprint of the computing device is valid.

9. The telemetry module of claim 5, wherein the software for certifying the validity of the instructions for programming the IMD is operable to determine whether vital signs are within an appropriate range for the instructions to be executed.

10. The telemetry module of claim 5, wherein the software for certifying the validity of the instructions for programming the IMD is operable to determine whether certain auxiliary equipment is present to provide safety for the execution of the instructions.

11. An implantable medical device (IMD) programming system comprising:
a computing device operable to generate and communicate programming instructions for programming an IMD; and
a telemetry module comprising:
telemetry electronics for communication with the implantable medical device (IMD);
an interface for communication with the computing device; and
a processing unit comprising software for controlling communications with the IMD and with the computer device via the telemetry electronics and the interface, respectively, for certifying the validity of the programming instructions communicated by the computing device, and for enabling performance of the programming instructions to program the IMD only after the instructions are certified as valid.

12. The IMD programming system of claim 11, wherein the interface of the telemetry module comprises a universal serial bus (USB) interface.

13. The IMD programming system of claim 11, wherein the telemetry module further comprises an input/output (I/O) interface for displaying information related to the status of the IMD and/or inputting programming instructions.

14. The IMD programming system of claim 11, wherein the telemetry module further comprises application software for executing the instructions for programming the IMD.

15. The IMD programming system of claim 11, wherein the software for certifying the validity of the instructions for programming the IMD executes an encryption scheme to certify the validity of the instructions.

16. A method of programming an implantable medical device (IMD) with a telemetry module, the method comprising:
generating programming instructions with a computing device;
establishing a communication session between the computing device and the telemetry module;
determining, at the telemetry module, whether the programming instructions generated by the computing device are valid for programming the IMD; and
upon certification of the programming instructions, operating the telemetry module to program the IMD according to the programming instructions.

17. The method of claim 16, wherein at least one of establishing a communication session between the computing device and the telemetry module and determining, at the telemetry module, whether the programming instructions generated by the computing device are valid for programming the IMD comprises:
generating a software fingerprint at the computing device;
transmitting the software fingerprint from the computing device to the telemetry module; and
determining, at the telemetry module, whether the software fingerprint is valid.

18. The method of claim 17, wherein transmitting the software fingerprint from the computing device to the telemetry module and determining, at the telemetry module, whether the software fingerprint is valid, involves an encryption scheme.

19. The method of claim 18, wherein upon determining that the software fingerprint is valid, a symmetric key is generated for encryption and decryption of future transmissions between the telemetry module and the computing device.

20. The method of claim 16, wherein determining, at the telemetry module, whether the programming instructions generated by the computing device are valid for programming the IMD comprises determining whether vital signs are within an appropriate range for the instructions to be executed.

21. The method of claim 16, wherein determining, at the telemetry module, whether the programming instructions generated by the computing device are valid for programming the IMD comprises determining whether certain auxiliary equipment is present to provide safety for the execution of the instructions.

22. An implantable medical device (IMD) programming system comprising:
a computing device executing an application program that is operable to generate and communicate programming instructions for programming an IMD, and to display information related to data retrieved from the IMD; and
a telemetry module comprising:
a telemetry processing unit connected to the computing device via a universal serial bus (USB) interface to receive power from the computing device and for communicative coupling to the computing device; and
a telemetry head connected to the telemetry processing unit for receiving transformed power to operate the telemetry head to wirelessly communicate with the IMD to interrogate the IMD so that data is retrieved and can be passed to the telemetry processing unit and on to the computing device, and to transmit signals for programming the IMD.

23. The IMD programming system of claim 22, wherein the application program executed by the computing device is stored in a medium that is physically blocked from being accessed without a tool.

24. The IMD programming system of claim 22, wherein the IMD is a pacemaker, and wherein the application program comprises an emergency key function that activates emergency pacing only when two buttons are simultaneously activated.

25. The IMD programming system of claim 22, wherein the data retrieved from the IMD is handled via Unicode resource DLLs that support the Chinese language.
